(19) United States
(12) Patent Application Publication
(10) Pub. No.: US 2023/0029648 A1
(43) Pub. Date: Feb. 2, 2023

(54) PLANAR END EFFECTOR WITH IRRIGATION

(71) Applicant: Biosense Webster (Israel) Ltd., Yokneam (IL)

(72) Inventors: Pieter Emmelius VAN NIEKERK, Rancho Santa Margarita, CA (US); Jamie Lynn HENRIQUEZ, Pasadena, CA (US); Cesar FUENTES-ORTEGA, Pasadena, CA (US); Carlo PAPPONE, Lecco (IT)

(73) Assignee: Biosense Webster (Israel) Ltd., Yokneam (IL)

(21) Appl. No.: 17/854,882

(22) Filed: Jun. 30, 2022

Related U.S. Application Data
(60) Provisional application No. 63/203,801, filed on Jul. 30, 2021.

(51) Int. Cl. A61B 5/287; A61B 5/00; A61B 18/00

(52) U.S. Cl. CPC A61B 5/287; A61B 5/6859; A61B 18/00; A61B 2018/00178

(57) ABSTRACT

Planar end effector designs having irrigation are presented. The example end effectors are configured to be affixed to a distal end of a catheter and delivered through vasculature in a collapsed configuration and expand at an intracardiac treatment site to a deployed configuration. In some instances, the end effector can have an electrode array with sufficient density to perform mapping and irrigation for mapping. The end effector can include dedicated irrigation tubes and/or irrigating electrode-carrying spines to irrigate within the electrode array. Flow rate at positions within the electrode array can be controlled in a predetermined manner by varying pore/port size, flow direction, and/or flow path cross-section throughout an irrigation flow path in the end effector.

### CROSS REFERENCE TO RELATED APPLICATIONS

This application claims the benefit of priority under 35 U.S.C. § 119 to prior filed U.S. Provisional Pat. Application No. 63/203,801 filed on Jul. 30, 2021, which is hereby incorporated by reference as set forth in full herein.

### FIELD

This invention relates to catheters, in particular, intravascular catheters for tissue diagnostics.

### BACKGROUND

Cardiac arrhythmia, such as atrial fibrillation, occurs when regions of cardiac tissue abnormally conduct electric signals to adjacent tissue, thereby disrupting the normal cardiac cycle and causing asynchronous rhythm. Sources of undesired signals are typically located in tissue of the atria a ventricle. Regardless of source, unwanted signals are conducted elsewhere through heart tissue where they can initiate or continue arrhythmia.

Procedures for treating arrhythmia include surgically disrupting the origin of the signals causing the arrhythmia, as well as disrupting the conducting pathway for such signals. More recently, it has been found that by mapping the electrical properties of the endocardium and the heart volume, and selectively ablating cardiac tissue by application of energy, it is possible to cease or modify the propagation of unwanted electrical signals from one portion of the heart to another. The ablation process destroys the unwanted electrical pathways by formation of non-conducting lesions.

### SUMMARY

Examples presented herein generally relate generally to planar end effector designs having irrigation. The example end effectors are configured to be affixed to a distal end of a catheter and delivered through vasculature in a collapsed configuration and expand at an intracardiac treatment site to a deployed configuration. The end effector can include dedicated irrigation tubes and/or irrigating electrode-carrying spines to irrigate within the electrode array. Flow rate at positions within the electrode array can be controlled in a predetermined manner by varying pore/port size, flow direction, and/or flow path cross-section throughout an irrigation flow path in the end effector. The end effector can have linear spine segments that are approximately planar but for overlap of connectors to the spine segments when the end effector is in the deployed configuration.

A first example end effector includes linear spine segments each carrying electrodes and a first irrigation tube distinct from the spine segments. The end effector is configured such that in the deployed configuration the spine segments are parallel to each other and approximately planar. The electrodes are positioned to define an electrode array when the end effector is in the deployed configuration. The first irrigation tube extends parallel to the spine segments and includes one or more central irrigation ports or pores positioned within the electrode array.

The first irrigation tube can further include a distal irrigation port positioned in a distal direction in relation to the electrode array and/or a proximal irrigation port positioned in a proximal direction in relation to the electrode array.

The end effector can include 4 to 6 spine segments.

The end effector can include a second irrigation tube and a third irrigation tube. The second and third irrigation tubes can extend parallel to the spine segments such that a spine segment is positioned between the first and second irrigation tubes and another spine segment is positioned between the first and third irrigation tubes. The second and third irrigation tubes can each include one or more central irrigation ports or pores positioned within the electrode array.

The end effector can include multiple loop members each including two of the spine segments and two ends affixed to a shaft of the catheter. A distal end of the first irrigation tube can be affixed to a distal segment of at least one of the plurality of loop members. When the end effector includes second and third irrigation tubes, a distal end of each of the respective irrigation tubes can be affixed to a respective distal loop member segment. The end effector can have exactly 2 or exactly 3 loop members.

The end effector can include an irrigation hub in fluidic communication with the first irrigation tube. The irrigation hub can be disposed in a distal direction in relation to the electrode array. The irrigation hub can include an irrigation port and an ingress port. The irrigation hub can be connected to each of the loop members. Each of the loop members can include irrigation pores along their respective length to allow fluid flow from the irrigation hub through the loop member in a proximal direction. The ingress port of the irrigation hub can be in fluidic communication with the first irrigation tube and the irrigation port can be in fluidic communication with one or more of the loop members.

The end effector can include irrigation holes on a proximal portion of the irrigation tube having a first diameter and irrigation holes on a distal portion the irrigation tube having a second diameter greater than the first diameter. The irrigation holes can be distributed longitudinally within the electrode array.

The electrodes of the end effector can each have a predetermined porosity to allow for irrigation fluid to flow out of each electrode.

A second example end effector includes linear spine segments configured such that when the end effector is in a deployed configuration the spine segments are parallel to each other and approximately planar. Each of the spine segments includes electrodes disposed over the respective spine segment, irrigation pores disposed between electrodes, and an irrigation lumen in fluidic communication with the respective irrigation pores.

Each of the linear spine segments can further include an elongated support member and electrode wires. The support member and wires can extend through the irrigation lumen. Alternatively, each of the linear spine segments can further include a second lumen parallel to the irrigation lumen and fluidically isolated from the irrigation lumen; the support member can extend through the irrigation lumen and the wires can extend through the second lumen. The irrigation lumen can have a cross-sectional area equal to or greater than a cross-sectional area of the second lumen. As another alternative, each of the linear spine segments can further include second and third lumens parallel to the irrigation lumen and fluidically isolated from the irrigation lumen and each other; the wires can extend through the second lumen; and the support member can extend through the third lumen.

The end effector can have 4 to 6 spine segments.

The end effector can include loop members each including two of the spine segments and two ends affixed to a shaft of the catheter.

The end effector can have exactly 2 or 3 loop members.

The end effector can further include an irrigation hub in fluidic communication with the respective irrigation lumen of at least a portion of the linear spine segments. The irrigation hub can be disposed in a distal direction in relation to the electrodes. The irrigation hub can include an irrigation port and an ingress port. The end effector can include an irrigation tube extending from a proximal portion of the end effector to a distal portion of the end effector and connecting to the irrigation hub thereby providing a flow path in a distal direction through the irrigation tube to the irrigation hub and a proximal direction from the irrigation hub through the loop members. The irrigation tube can connect to the ingress port of the irrigation hub.

The end effector can include irrigation holes on a proximal portion of the irrigation tube having a first diameter and irrigation holes on a distal portion the irrigation tube having a second diameter greater than the first diameter. The irrigation holes can be distributed longitudinally within the electrode array.

The electrodes of the end effector can each have a predetermined porosity to allow for irrigation fluid to flow out of each electrode.

A third example end effector includes linear spine segments each carrying electrodes thereon to define an electrode array, a central irrigation lumen extending into the electrode array, and branch irrigation lumens extending proximally from a distal end of the central irrigation lumen. The end effector is configured such that when deployed, the spine segments are parallel to each other and approximately planar. The central irrigation lumen is configured to deliver fluid in a distal direction. The branch irrigation lumens are configured to deliver fluid in a proximal direction. The central lumen and/or one or more of the branch irrigation lumens can extend through the spine segments. Additionally, or alternatively, the central lumen and/or one or more of the branch irrigation lumens can extend through an irrigation tube distinct from the spine segments.

The central irrigation lumen can have a cross-sectional area greater than a respective cross-sectional area of each of the branch irrigation lumens.

The end effector can further include an irrigation hub disposed at a distal end of the end effector. The irrigation hub can provide a flow path from the central irrigation lumen to each of the branch irrigation lumens.

The electrodes can each have a predetermined porosity to allow for irrigation fluid to flow out of each electrode.

The end effector can further include irrigation holes on a proximal portion of the irrigation tube having a first diameter and irrigation holes on a distal portion the irrigation tube having a second diameter greater than the first diameter.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example catheter according to aspects of the present invention.

FIGS. 2A and 2B are illustrations of example end effectors having a central irrigation tube according to aspects of the present invention.

FIGS. 3A and 3B are illustrations of example end effectors having three irrigation tubes according to aspects of the present invention.

FIG. 4 is an illustration of another example end effector having a central irrigation tube affixed at a distal end to loop members according to aspects of the present invention.

FIG. 5 is an illustration of another example end effector having three irrigation tubes each affixed at their respective distal ends to loop members according to aspects of the present invention.

FIG. 6A is an illustration of another example end effector having irrigating spine segments according to aspects of the present invention.

FIGS. 6B through 6E are illustrations of alternative cross-sections of the spine segment of the example end effector as indicated in FIG. 6A.

FIG. 7 is an illustration of another example end effector having a proximal flow path through spines according to aspects of the present invention.

FIG. 8 is an illustration of an example porous electrode according to aspects of the present invention.

### DETAILED DESCRIPTION

The following description of certain examples of the invention should not be used to limit the scope of the present invention. The drawings, which are not necessarily to scale, depict selected embodiments and are not intended to limit the scope of the invention. The detailed description illustrates by way of example, not by way of limitation, the principles of the invention. Other examples, features, aspects, embodiments, and advantages of the invention will become apparent to those skilled in the pertinent art from the following description, which includes one of the best modes contemplated for carrying out the invention. As will be realized, the invention is capable of other different or equivalent aspects, all without departing from the invention. Accordingly, the drawings and descriptions should be regarded as illustrative in nature and not restrictive.

Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the pertinent art in view of the teachings herein. Such modifications and variations are intended to be included within the scope of the claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “patient,” “host,” “user,” and “subject” refer to any human or animal subject and are not intended to limit the systems or methods to human use, although use of the subject invention in a human patient represents a preferred embodiment.

When used herein, the terms “tubular” and “tube” are to be construed broadly and are not limited to a structure that is a right cylinder or strictly circumferential in cross-section or of a uniform cross-section throughout its length. Tubular structures are generally illustrated herein as substantially right cylindrical structures. However, the tubular structures may have a tapered or curved outer surface without departing from the scope of the present invention.

When used herein, the terms “port” and “pore” indicate an opening providing a passageway for fluid egress. Both “port” and “pore” can be used respectively to refer to an opening that is singular or distributed among like sized openings. The terms are generally interchangeable. The term “port” is used herein to describe an opening that is preferably singular or distributed among a few other like sized openings or ports but the term is not strictly limited as such. The term “pore” is used herein to describe an opening that is preferably distributed among several other like sized openings or pores but the term is not strictly limited as such.

Examples presented herein generally relate to end effector designs having irrigation from, and/or among, electrode-carrying linear spines. Each of the example end effectors are configured to be affixed to a distal end of a catheter, delivered through vasculature in a collapsed configuration, and expand at an intracardiac treatment site to a deployed configuration in which the linear spines are parallel to each other and approximately coplanar to each other. The spines can be coplanar but for overlap of connecting members of the end effector which join the spines at a proximal end to the shaft and/or at a distal end to other spines. The spines preferably become coplanar when the end effector 100 is pressed to a planar surface, where one example of such a configuration is described in U.S. Pat. Application 17/029,752, now U.S. Publication No. 2021/0369339 A1, which is hereby incorporated by reference in its entirety into this application as if set forth in full and attached in the appendix to priority application U.S. 63/203,801.

Examples illustrated herein include 4 or 6 spines, which are the presently preferred number of spines; however other numbers of spines such as 3, 5, 7, 8, 9, or 10 linear spines are within the scope of the present invention by modification of examples presented herein according to practices known to persons skilled in the pertinent art. Spine electrodes can be arranged in an electrode array in which the electrodes are regularly or irregularly distributed across the end effector when deployed.

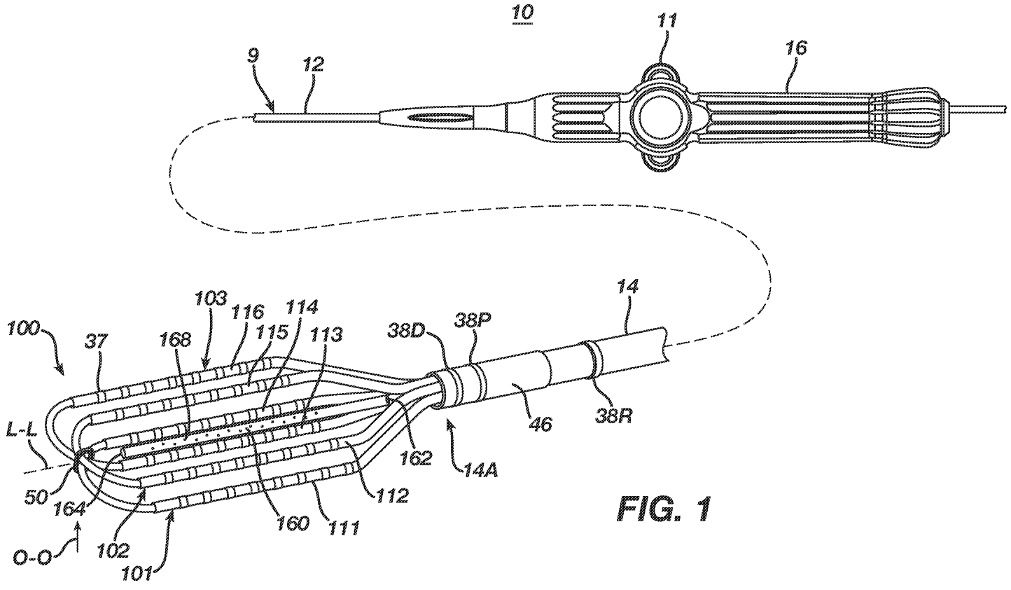

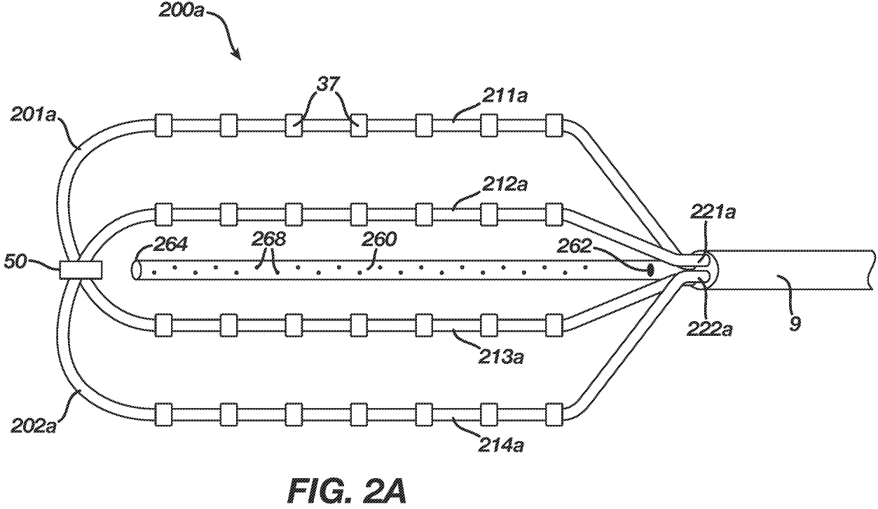

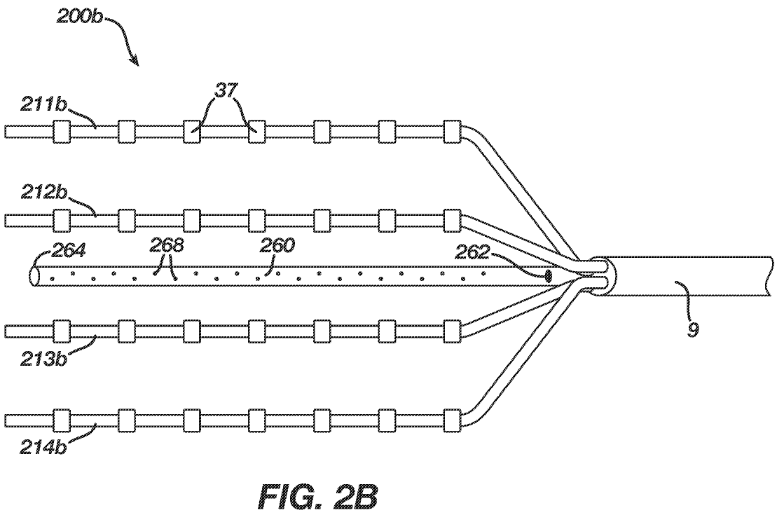

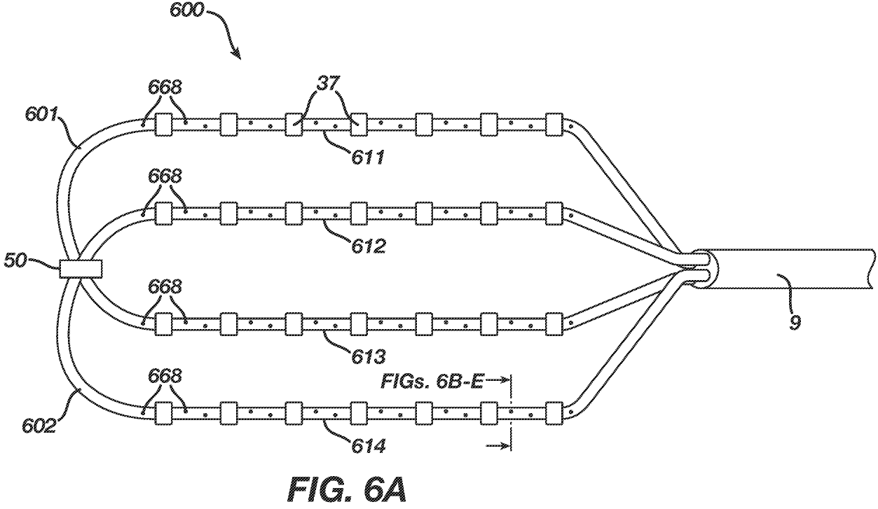

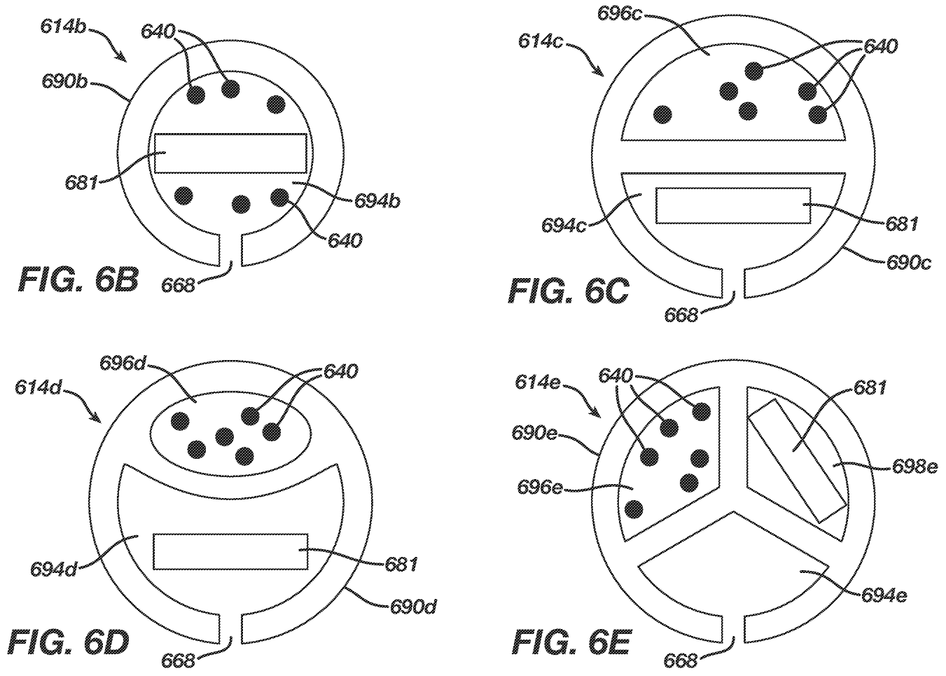

The end effector can include pores/ports configured to provide irrigation fluid in the vicinity of the electrode array during a treatment procedure. The pores/ports can be disposed on the spines (e.g. FIGS. 6A, 7, and 8) and/or on one or more dedicated irrigation tubes traversing the electrode array (e.g. FIGS. 1, 2A-B, 3A-B, 4, and 5). Spines having irrigation pores can have several lumen configurations (e.g. FIGS. 6A-6E). Examples illustrated herein which include irrigation tubes include 1 or 3 irrigation tubes, which are presently the preferred number of irrigation tubes; however other numbers of irrigation tubes such as 2, 4, 5, 6, 7, 8, or 9 are within the scope of the present invention by modification of examples presented herein according to practices known to persons skilled in the pertinent art.

The pores/ports can be, sized, shaped, distributed, and otherwise configured to provide a predetermined flow rate. In some examples, pores/ports which are closer to the source of the fluid, and therefore experience higher fluid pressure, can be sized smaller than pores/ports that are further downstream and therefore have a lower fluid pressure so that the flow rate is more uniform across the pores/ports than if the pores/ports were all equally sized. Additionally, or alternatively, pores/ports having approximately equal size can be more densely distributed downstream to provide a more uniform flow rate than if the pores/ports were evenly distributed. In some examples pores can be sized, positioned, and otherwise configured to provide flow to inhibit blood stagnation and thrombosis in the vicinity of the end effector.

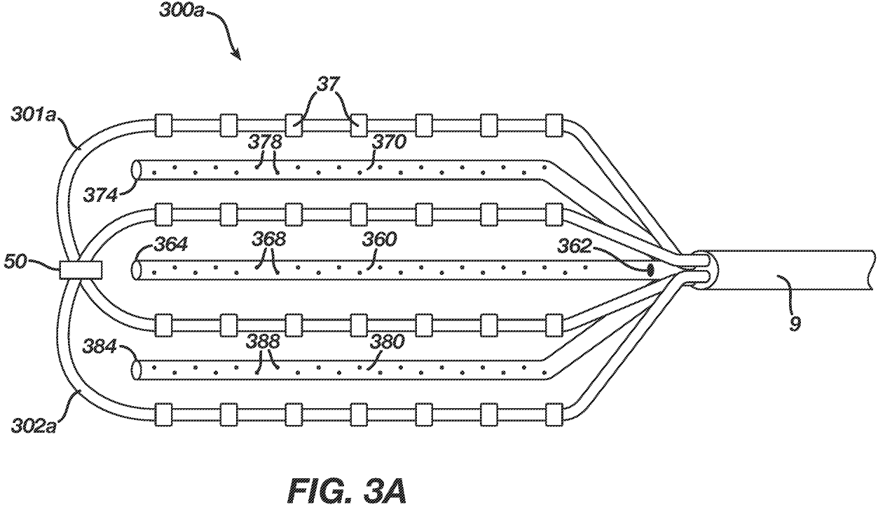

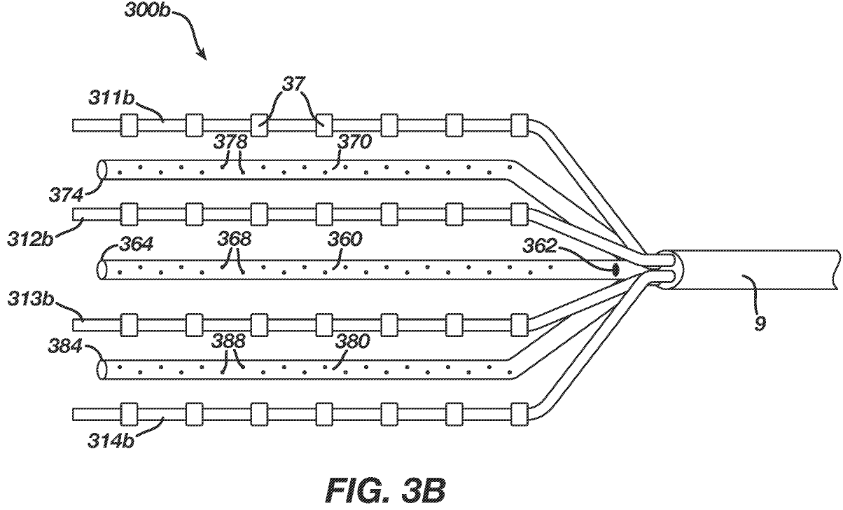

Examples presented herein include some example end effectors having loop members each including two of the linear spines connected by a curved distal loop segment (e.g. FIGS. 1, 2A, 3A, 4, 5, 6A, and 7) and other example end effectors having a forked design in which the linear spines have free distal ends (e.g. FIGS. 2B and 3B). Example end effectors can be modified to include a combination of both loop members and linear spines with free ends. Example end effectors can be modified with additional structures such as connectors at midpoints of the linear spines similar to as described in U.S. Pat. 10,537,259 which is hereby incorporated by reference in its entirety into this application as if set forth in full and attached in the appendix to priority application U.S. 63/203,801. Example end effectors can be modified to include compatible structures described in U.S. Pat. 7,366,557, 9,820,664, 9,949,656, and 10,537,259 and U.S. Pat. Pub. 2020/0038101 each of which is hereby incorporated by reference in its entirety into this application as if set forth in full and attached in the appendix to priority application U.S. 63/203,801.

FIG. 1 illustrates an example apparatus 10 having an elongated tubular shaft 9, a distal electrode assembly or end effector 100, and a deflection control handle 16. The elongated shaft 9 has a proximal portion 12 in the shape of an elongated catheter body, an intermediate deflection section 14, and distal portion 14A. The deflection control handle 16 is attached to the proximal end of the catheter body 12. The distal portion 14A of the shaft 9 is coupled to the end effector 100 via a connector tubing 46. The elongated shaft 9 forms a tubular catheter body sized and otherwise configured to traverse vasculature.

The end effector 100 has a plurality of loop members 101, 102, 103 that overlap at a common distal vertex and are joined at the distal vertex with a mechanical linkage 50. Each loop member 101, 102, 103 includes two linear spine segments 111, 112, 113, 114, 115, 116 having electrodes 37 thereon. The end effector 100 can include a central irrigation tube 160 distinct from the spine segments 111, 112, 113, 114, 115, 116. The central irrigation tube 160 can have central pores 168 distributed longitudinally along the central irrigation tube 160 and within an electrode array defined by the electrodes 37. As illustrated, the electrode array is a grid including all of the electrodes 37 on the end effector 100, and the electrodes 37 on the end effector 100 are regularly spaced. Alternatively, the electrodes 37 can be irregularly spaced and define an electrode array.

The irrigation tube 160 is also illustrated as having a proximal irrigation port 162 located in a proximal direction in relation to the electrode array and a distal irrigation port 164 located in a distal direction in relation to the electrode array. Irrigation fluid can be pumped into the handle 16, through the shaft 9, into the irrigation tube 160 and out through the central pores 168, proximal irrigation port 162, and/or distal irrigation port 164 to provide irrigation to tissue. The pores 168 of the irrigation tube 160 can be sized, shaped, distributed, or otherwise configured to provide a uniform flow rate longitudinally across the irrigation tube 160. For instance, pores 168 on a proximal portion of the central irrigation lumen can have a smaller diameter than pores 168 on a distal portion of the central irrigation tube 160.

When the device is unconstrained and aligned, the proximal portion 12, intermediate section 14, distal portion 14A, and end effector 100 are generally aligned along a longitudinal axis L-L. The intermediate section 14 can be configured to bend to deflect the distal portion 14A and end effector 100 from the longitudinal axis L-L.

The end effector 100 can be collapsed (compressed toward the longitudinal axis L-L) to fit within a guiding sheath or catheter (not illustrated). The shaft 9 can be pushed distally to move the end effector 100 distally through the guiding sheath. The end effector 100 can be moved to exit a distal end of the guiding sheath via manipulation of the shaft 9 and/or control handle 16. An example of a suitable guiding sheath for this purpose is the Preface Braided Guiding Sheath, commercially available from Biosense Webster, Inc. (Irvine, California, USA).

For each loop member 101, 102, 103 the spines 111, 112, 113, 114, 115, 116 can be substantially parallel to each other along a majority of their respective lengths when the end effector 100 is expanded in an unconstrained configuration as illustrated in FIG. 1. The spines can be approximately coplanar, where deviations in being coplanar are a result of overlap of the loop members 101, 102, 103 at the distal linkage 150 and/or at ends connected to the shaft 9. The spines can be approximately coplanar in a plane perpendicular to an orthogonal axis O-O as illustrated in FIG. 1. The loop members 101, 102, 103 can be configured such that the spines 111, 112, 113, 114, 115, 116 become coplanar when the end effector 100 is pressed to a planar surface similar to as described in U.S. Pat. Application 17/029,752, now U.S. Publication No. 2021/0369339 A1, which is hereby incorporated by reference in its entirety into this application as if set forth in full and attached in the appendix to priority application U.S. 63/203,801. The central tube 160 can also be approximately coplanar to the spines 111, 112, 113, 114, 115, 116 as illustrated.

Each spine segment can have a length ranging between about 5 and 50 mm, preferably about 10 and 35 mm, and more preferably about 28 mm. The spine segments can be spaced apart from each other by a distance ranging between about 1 mm and about 20 mm, preferably between about 2 and about 10 mm, and more preferably about 4 mm. Each spine preferably carries at least eight electrodes per spine member. With eight electrodes 37 on six spines 111, 112, 113, 114, 115, 116, the end effector 100 includes forty-eight electrodes 37 in the end effector electrode array.

A distal electrode 38D and a proximal electrode 38P are positioned near the distal portion 14A of the shaft 9. These electrodes 38D, 38P can be configured to cooperate (e.g. by masking of a portion of one electrode and masking a different portion on the other electrode) to define a referential electrode (an electrode that is not in contact with tissues). One or more impedance sensing electrodes 38R can be configured to allow for location sensing via impedance location sensing technique, as described in U.S. Pat. Nos. 5,944,022; 5,983,126; and 6,445,864, each of which is hereby incorporated by reference in its entirety into this application as if set forth in full and attached in the appendix to priority application U.S. 63/203,801. Electrodes 38, 38P, 38R on the shaft 9 are not considered a part of the end effector electrode array because they are positioned on the shaft 9 rather than on the end effector 100.

FIGS. 2A and 2B are illustrations of example end effectors 200a, 200b having a central irrigation tube 260. The end effector 200a illustrated in FIG. 2A is similar to that illustrated in FIG. 1 with a difference being that the end effector 200a of FIG. 2A includes two loop members 201a, 202a and four linear spine segments 211a, 212a, 213a, 214a rather than rather than three loop members 101, 102, 103 and six linear spine segments 111, 112, 113, 114, 115. The end effector 200b illustrated in FIG. 2B includes four linear spine segments 211b, 212b, 213b, 214b lacking distal connecting segments to form loop members 201a, 202a illustrated in FIG. 2A.

The end effectors 200a, 200b illustrated in FIGS. 2A and 2B each include a central irrigation tube 260 having central irrigation pores 268, a proximal irrigation port 262, and a distal irrigation port 264 similar to the irrigation tube 160 illustrated in FIG. 1. Each of the spine segments 211a-b, 212a-b, 213a-b, 214a-b can be approximately coplanar, i.e. coplanar but for overlap of connecting segments. The central irrigation tube 260 can also be approximately coplanar with the spine segments. As illustrated in FIG. 2A, each loop member (including those illustrated elsewhere) can include two ends 221a, 222a affixed to the shaft 9 at a distal end of the shaft 9.

As illustrated, each linear spine segment 211a-b, 212a-b, 213a-b, 214a-b carries seven electrodes 37 for a total of twenty-eight electrodes 37 in the electrode array of the end effector 100.

FIGS. 3A and 3B are illustrations of example end effectors having three irrigation tubes 360, 370, 380. Each irrigation tube 360, 370, 380 respectively includes central irrigation pores 368, 378, 388 positioned within an electrode array defined by the electrodes 37 of the end effector 300a, 300b. Each irrigation tube 360, 370, 380 includes a respective distal irrigation port 364, 374, 384. The central irrigation tube 360 includes a proximal irrigation port 362. The loop members 301a, 302a of the end effector 300a illustrated in FIG. 3A can be configured similarly to the loop members 201a, 202a of the end effector 200a illustrated in FIG. 2A. The linear spine segments 311b, 312b, 313b, 314b of the end effector 300b illustrated in FIG. 3B can be configured similarly to the linear spine segments 211b, 212b, 213b, 214b of the end effector 200b illustrated in FIG. 2B. The irrigation tubes 360, 370, 380 can be interleaved in between linear spine segments 311a-b, 312a-b, 313a-b as illustrated. The irrigation tubes 360, 370, 380 can be approximately coplanar to the spine segments 311a-b, 312a-b, 313a-b.

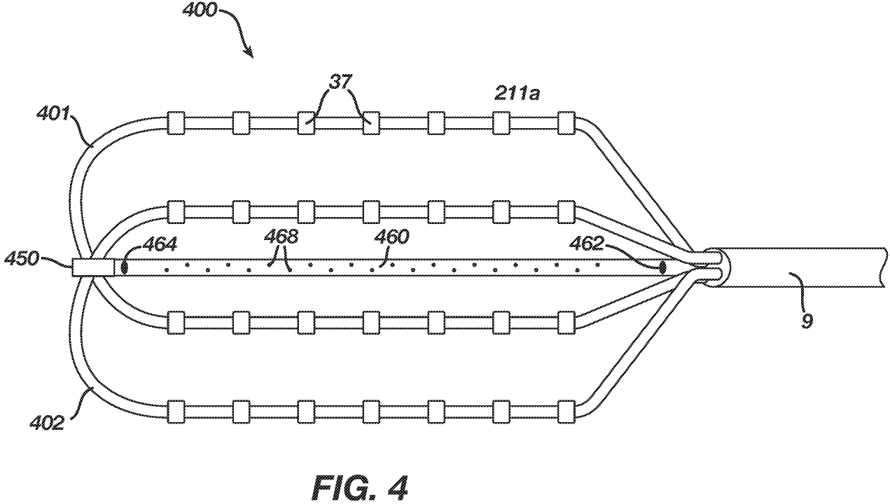

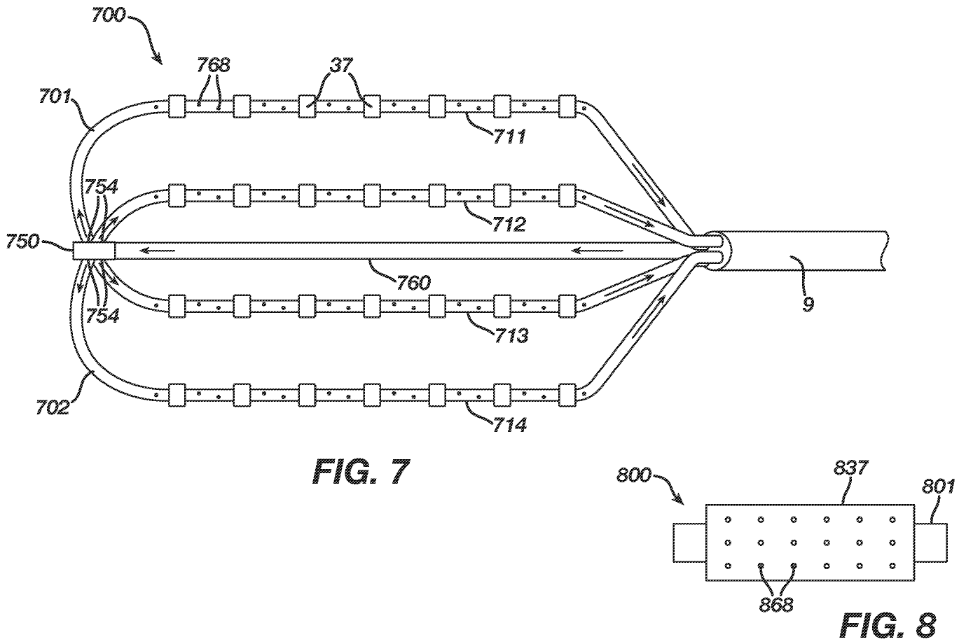

FIG. 4 is an illustration of another example end effector 400 having central irrigation tube 460 affixed at a distal link 450 to loop members 401, 402. The connection to the distal link 450 can facilitate movement of the central irrigation tube 460 in concert with the loop members 401, 402 as the end effector 400 flexes. The central tube 460 can include central pores 468 distributed longitudinally through the electrode array. The central tube 460 can include a proximal irrigation port 462 and a distal irrigation port 464. The distal link 450 can further be configured to function as an irrigation hub similar to the irrigation hub 750 illustrated in FIG. 7.

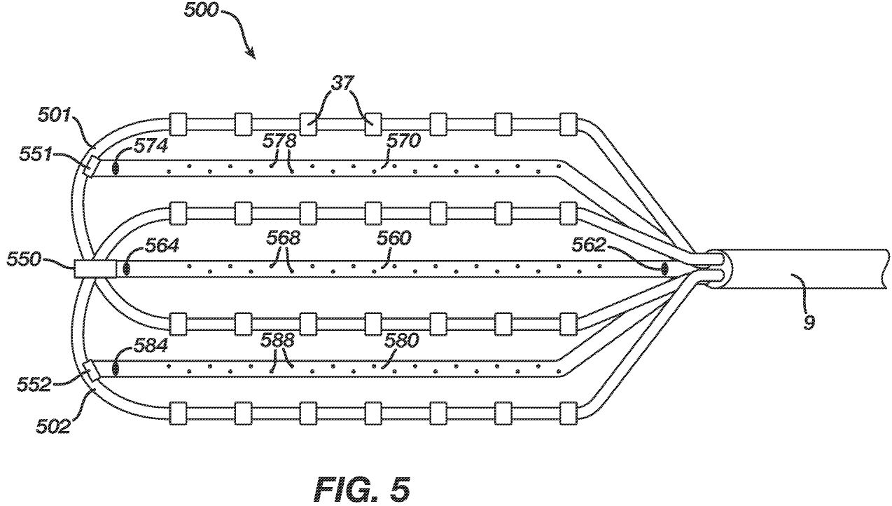

FIG. 5 is an illustration of another example end effector 500 having three irrigation tubes 560, 570, 580 each affixed at their respective distal ends with respective mechanical linkages 550, 551, 552 to loop members 501, 502. The irrigation tubes 560, 570, 580 can be interleaved between linear spine segments. The connection to the irrigation tubes 560, 570, 580 to the distal mechanical linkages 550, 551, 552 can facilitate movement of the irrigation tubes 560, 570, 580 in concert with the loop members 501, 502 as the end effector 500 flexes. Each irrigation tube can include central pores 568, 578, 588 and a distal irrigation port 564, 574, 584. The central tube 560 can further include a proximal irrigation port 562. Some or all of the mechanical linkages 550, 551, 552 can be configured to function respectively as an irrigation hub similar to the irrigation hub 750 illustrated in FIG. 7. The end effector 500 can be modified include a combination of irrigation tubes linked to one or more loop members at a distal linkage and one or more irrigation tubes having a free distal end such as illustrated in FIG. 3B.

FIG. 6A is an illustration of another example end effector 600 having loop members 601, 602 with irrigating spine segments 611, 612, 613, 614 with pores 668 therethrough. Each spine segment 611, 612, 613, 614 can have an irrigation lumen therethrough in fluidic communication with the pores 668. Pores 668 can be distributed between electrodes 37.

FIGS. 6B through 6E are illustrations of alternative cross-sections of the spine segment of the example end effector 600 as indicated in FIG. 6A.

FIG. 6B illustrates a cross-section of a first example spine segment 614b including a tube 690b having a single lumen 694b which houses wires 640 connecting to electrodes 37 and a support member 681 as well as providing irrigation. The tube 690b includes a pore 668 at the cross-section to allow fluid to flow from the irrigation lumen 694b.

FIG. 6C illustrates a cross-section of a second example spine segment 614c including a tube 690c having an irrigation lumen 694c and a wire lumen 696c. The irrigation lumen 694c includes a support member 681 extending therethrough. The wire lumen 696c includes wires 640 to the electrodes 37 extending therethrough. The two lumens 694c, 696c are fluidically isolated from each other. The two lumens 694c, 696c are illustrated as being approximately equal in cross sectional area.

FIG. 6D illustrates a cross-section of a third example spine segment 614d including a tube 690d having an irrigation lumen 694d fluidically isolated from a wire lumen 696d in which the wire lumen 696d has a smaller cross sectional area than the irrigation lumen 694d. A support member 681 is illustrated extending through the irrigation lumen 694d.

FIG. 6E illustrates a cross-section of a fourth example spine segment 614e including a tube 690e having an irrigation lumen 694e, a wire lumen 696e, and a support member lumen 698e. The lumens 694e, 696e, 698e are fluidically isolated from each other, separating wires 640, support member 681, and fluid in the irrigation lumen 694e.

FIG. 7 is an illustration of another example end effector 700 having a proximal flow path through spines 711, 712, 713, 714 having irrigation pores 768. The end effector 700 includes an irrigation tube 760 having an irrigation lumen to provide a flow path in the distal direction. The end effector 700 includes an irrigation hub 750 connecting to a distal end 752 of the irrigation tube 760. The irrigation hub 750 can have one or more inlets connected to the irrigation tube 760 and one or more outlets 754 the loop members 701, 702 to provide one or more flow paths from the irrigation tube 760 to the loop members 701, 702. Fluid from the irrigation tube 760 can flow proximally from the irrigation hub 750 through the linear spine segments 711, 712, 713, 714. The central irrigation tube 760 can have an irrigation lumen that has a greater cross section than either of the branch irrigation lumens which flow through the linear spine segments 711, 712, 713, 714.

FIG. 8 is an illustration of an example porous electrode 837 on a linear spine segment 801 of another example end effector 800. As illustrated the electrode 837 includes pores 868 in fluidic communication with an irrigation lumen such as illustrated in FIGS. 6B-6E or otherwise configured to irrigate as understood by persons skilled in the pertinent art, e.g. as described in U.S. Pat. 10,537,259 which is hereby incorporated by reference in its entirety into this application as if set forth in full and attached in the appendix to priority application U.S. 63/203,801. Porosity of the electrodes can be predetermined to provide a desired flow rate during treatment.

Features of each of the examples described and illustrated herein can be combined and modified as understood by persons skilled in the pertinent art according to the teachings herein. Pores/ports can be added to spine segments and irrigation tubes which are illustrated or described as lacking pores/ports. Pores/ports can be removed selectively and/or entirely from some of the spines or tubes. Pores/ports can vary in size, shape, distribution density, or otherwise be configured to provide a predetermine flow rate corresponding to variations in pressure through flow paths of the end effector. Irrigation lumens can vary in cross sectional area to modulate pressure and/or flow rate through the flow paths of the end effector. Additional mechanical support structures and/or sensors can be added to the end effectors. Examples presented herein are therefore illustrative and are not intended to be limiting.

What is claimed is:

1. An end effector at a distal end of a catheter and being configured to be delivered through vasculature in a collapsed configuration and expand at an intracardiac treatment site to a deployed configuration, the end effector comprising:
a plurality of linear spine segments each carrying electrodes thereon and configured such that in the deployed configuration the spine segments are parallel to each other and approximately planar such that the electrodes are positioned to define an electrode array; and
a first irrigation tube distinct from the spine segments, extending parallel to the spine segments, and comprising a first central irrigation port positioned within the electrode array.

2. The end effector of claim 1, the first irrigation tube comprising a plurality of pores comprising the first central irrigation port and distributed longitudinally through the electrode array on the first irrigation tube.

3. The end effector of claim 1, the first irrigation tube further comprising a distal irrigation port positioned in a distal direction in relation to the electrode array.

4. The end effector of claim 1, the first irrigation tube further comprising a proximal irrigation port positioned in a proximal direction in relation to the electrode array.

5. The end effector of claim 1, the end effector further comprising:
a plurality of loop members each comprising two of the spine segments and two ends affixed to a shaft of the catheter.

6. The end effector of claim 5, a distal end of the first irrigation tube being affixed to a distal segment of at least one of the plurality of loop members.

7. The end effector of claim 1, the end effector further comprising:
a second irrigation tube extending parallel to the spine segments such that at least one spine segment of the linear spine segments is positioned between the first and second irrigation tubes, the second irrigation tube comprising a second central irrigation port; and
a third irrigation tube extending parallel to the spine segments such that at least one spine segment of the linear spine segments is positioned between the first and third irrigation tubes, the third irrigation tube comprising a third central irrigation port.

8. The end effector of claim 7, the end effector further comprising:
a plurality of loop members each comprising two of the spine segments and two ends affixed to a shaft of the catheter,
wherein a distal end of the first irrigation tube is affixed to a distal segment of at least one of the plurality of loop members,
wherein a distal end of the second irrigation tube is affixed to a distal segment of at least one of the plurality of loop members, and
wherein a distal end of the third irrigation tube is affixed to a distal segment of at least one of the plurality of loop members.

9. The end effector of claim 1, further comprising:
an irrigation hub in fluidic communication with the first irrigation tube, disposed in a distal direction in relation to the electrode array, and comprising an irrigation port and an ingress port.

10. The end effector of claim 9, the end effector further comprising:
a plurality of loop members each comprising two of the spine segments and two ends affixed to a shaft of the catheter,
the irrigation hub being connected to each of the loop members, and
each of the loop members comprising irrigation pores along their respective length to allow fluid flow from the irrigation hub through the loop member in a proximal direction.

11. The end effector of claim 1, further comprising:
a first plurality of irrigation holes on a proximal portion of the irrigation tube each comprising a first diameter; and
a second plurality of irrigation holes on a distal portion the irrigation tube distal to the proximal portion each comprising a second diameter greater than the first diameter.

12. The end effector of claim 1, the electrodes each comprising a predetermined porosity to allow for irrigation fluid to flow out of each electrode.

13. An end effector at a distal end of a catheter and being configured to be delivered through vasculature in a collapsed configuration and expand at an intracardiac treatment site to a deployed configuration, the end effector comprising:
a plurality of linear spine segments configured such that in the deployed configuration the spine segments are parallel to each other and approximately planar, each of the linear spine segments comprising:
respective electrodes disposed over the respective spine segment;
respective irrigation pores disposed between electrodes; and
a respective irrigation lumen in fluidic communication with the respective irrigation pores.

14. The end effector of claim 13, each of the linear spine segments further comprising:
a respective elongated support member extending through the respective irrigation lumen;
a respective second lumen parallel to the respective irrigation lumen and fluidically isolated from the respective irrigation lumen; and
respective electrode wires extending through the respective second lumen.

15. The end effector of claim 13, each of the linear spine segments further comprising:
a respective second lumen parallel to the respective irrigation lumen and fluidically isolated from the respective irrigation lumen;
respective electrode wires extending through the respective second lumen;
a respective third lumen parallel to the respective irrigation lumen and fluidically isolated from the respective irrigation lumen and the respective second lumen; and
a respective elongated support member extending through the respective third lumen.

16. The end effector of claim 13, further comprising:
an irrigation hub in fluidic communication with the respective irrigation lumen of at least a portion of the linear spine segments, being disposed in a distal direction in relation to the electrodes and comprising an irrigation port and an ingress port.

17. The end effector of claim 13, the plurality of linear spine segments consisting of 4 to 6 spine segments.

18. An end effector at a distal end of a catheter and being configured to be delivered through vasculature in a collapsed configuration and expand at an intracardiac treatment site to a deployed configuration, the end effector comprising:
a plurality of linear spine segments each carrying electrodes thereon and configured such that in the deployed configuration the spine segments are parallel to each other and approximately planar such that the electrodes are positioned to define an electrode array; and
a central irrigation lumen extending into the electrode array and configured to deliver fluid in a distal direction; and
a plurality of branch irrigation lumens extending proximally from a distal end of the central irrigation lumen and configured to deliver fluid in a proximal direction.

19. The end effector of claim 18, the central irrigation lumen comprising a cross-sectional area greater than a respective cross-sectional area of each of the branch irrigation lumens.

20. The end effector of claim 18, further comprising:
a first plurality of irrigation holes providing outlets from a proximal portion of the central irrigation lumen, each of the first plurality of irrigation holes comprising a first diameter; and
a second plurality of irrigation holes providing outlets from a distal portion the central irrigation lumen distal to the proximal portion, each of the second plurality of irrigation holes comprising a second diameter greater than the first diameter.
